(19) United States
(12) Patent Application Publication
(10) Pub. No.: US 2024/0164421 A1
(43) Pub. Date: May 23, 2024

(54) NATURALLY FERMENTED COMPOSITION OF POLLEN USING GLACIAL SNOW WATER AND PREPARATION METHOD THEREFOR

(71) Applicants: Byong Chul YOO, Jeonju-si (KR); Mi Ryeong KIM, Jeonju-si (KR); Feng He JIN, Jilin City (CN)

(72) Inventors: Byong Chul YOO, Jeonju-si (KR); Mi Ryeong KIM, Jeonju-si (KR); Feng He JIN, Jilin City (CN)

(21) Appl. No.: 18/426,079

(22) Filed: Jan. 29, 2024

Related U.S. Application Data
(62) Division of application No. 16/980,249, filed on Sep. 11, 2020, now abandoned.

(30) Foreign Application Priority Data
Mar. 12, 2018 (KR) 10-2018-0028710

(51) Int. Cl. A23L 21/20; A23L 2/38; A23L 2/52; A23L 33/105; A61K 36/18

(52) U.S. Cl. CPC A23L 21/20; A23L 2/382; A23L 2/52; A23L 33/105; A61K 36/18; A23V 2002/00

(57) ABSTRACT

The present invention relates to a natural fermented composition of pollen using glacial snow water obtained by mixing pollen, such as bee pollen, pollen, and edible flowers, with glacial snow water and fermenting same, and a preparation method therefor. In the present invention, provided is a composition obtained by mixing glacial snow water having a pH of 7.5 to 8.8 and a density of 0.05-0.85 g/cm³ with one or more kinds of pollens selected from bee pollen, pollen, flower buds, floral leaves, and floral nectaries and fermenting the mixture for three months or longer without inoculation of microorganisms. The natural fermented composition of the pollens of the present invention contains a wide variety of nutritional and active ingredients, and can exhibit various effects. Especially, since the composition has excellent antioxidative effects, the composition may be used for foods and beverages, antioxidant drinks, etc.

### TECHNICAL FIELD

The present invention relates to a naturally fermented composition of pollen using glacial snow water obtained by mixing pollen such as bee pollen, other pollens and edible flowers, with glacial snow water and fermenting the same, as well as a preparation method thereof.

### BACKGROUND ART

Pollen is a germ cell from stamens of flowers blooming in vegetation and flowering plants and has a very small size of 0.1 to 0.003 mm. The husk of pollen is not well molten in aqua regia or hydrogen fluoride. Therefore, the pollen should be milled or crushed by a machine, or the husk thereof must be removed by applying physical or mechanical force. Pollen used as a health functional food refers to a product obtained by removing foreign substances from the pollen collected from bees or artificially collected pollen and then shredding the husks. Further, pollen extract refers to a concentrate or powder obtained by mechanically crushing or treating the husk of the pollen with enzymes, extracting the treated product, and then concentrating or drying the extract. Pollen is a food source for honeybees and is called a perfect food in Europe because of its high nutritional value. Small particles of pollen contain all the nutrients needed for life and growth. Ingredients in pollen specifically include 0.1 to 22% of carbohydrate, 0.1 to 19% of reductive sugar, 7 to 35% of protein, 10% of amino acid, 3 to 4% of moisture, etc., and active ingredients may specifically include vitamin A, vitamin B group, vitamin C and vitamin P (rutin), as well as minerals such as calcium, potassium, phosphorous, copper, magnesium, iron, silicon, manganese, sulfur, etc.

Bee pollen, in particular, refers to pollen collected by bees and is also known as a perfect food containing almost all of the nutrients needed by the human body. More particularly, bee Pollen contains three major nutrients (carbohydrates, proteins, fats), vitamins (18 species), minerals (25 species), amino acids (22 species), enzymes (11 species), fatty acids (14 species), and more than 50 bioactive substances. Further, it has been found to have excellent effects on anti-oxidation, improvement of immunity, strengthening of physical strength, and recovery from fatigue. Further, several types of flavonoids with very high antibacterial properties not found in other plants are also contained. Bee pollen contains about 5% of organic substance and minerals, among which flavonoids as well as minerals or vitamins, amino acid and fat components playing an important role in cell metabolism are included in high concentrations. Therefore, pollen has excellent antioxidant effects to prevent active oxygen in the body while improving health. Further, the pollen stimulates NK cells to increase immunity, and contains vitamin P and quercetin-rhamnose glycosides to thus strengthen blood vessels, thereby preventing capillaries from weakening.

Such pollen has been known as food for beauty, vigorousness and longevity. Hippocrates also used honey and pollen for medical treatment. Further, pollen has been used for health promotion in Europe and the United States from 30-40 years ago, and has also been commercially available as health food in Japan from about 20 years ago. In addition, pollen was used as a folk remedy for wound and ulcer treatment, antibacterial treatment, improvement of respiratory disease, facilitated digestion and absorption, intestinal function control, muscle vitality, improvement of renal and bladder diseases, rejuvenation, etc. Moreover, rats bred with pollen feed in animal experiments were reported to have good development and increased fertility.

Currently, pollen is commonly used as formulated health food for intake or may be ingested by a method of removing the husk of pollen and adding the product to honey, yogurt, salad, so on. However, these ingestion methods are less effective in digestive absorption, and many people avoid pollen husks since the husk may contain some ingredients acting as a source of allergens in the body.

Flowers and fruits are known to have a much higher content of active ingredients and nutrients compared to leaves and roots of plants, but are not used properly. In particular, although plant parts such as flowers, buds, petals and honey glands of flowers include pollen having such efficacies as described above, these are not well utilized.

Flower honey gland is a sugar organ, that is, an organ secreting honey, and sometimes attracts insects and birds to act as a pollinator. An inner honey gland of flower is present inside the flower, in particular, the ovary (i.e., the upper part of the ovary in the flower having upper ovary) or the stamen, attracts insects or birds to act as a pollinator. On the other hand, an outer honey gland of flower exists in the stem or leaf such as the upper part of a leaf stalk in cherry blossom, the upper part of a leaf body in Mallotus japonicus, etc. Since the honey gland is the organ secreting sugar-containing mucus from flowers, this substantially contains some nutrients and active ingredients to produce mucus.

Fruit peels are also known to contain much more nutrients including health antioxidants compared to flesh. As well as apples, grapes, etc., which are well known to people, the skins of kiwi, banana, watermelon, melon, etc., which are not commonly eaten, contain many nutrients and have been reported to show advantageous effects on the human body. Melting water of glaciers (“glacial snow water”) is the melting water from firn (or neve), glacier snow, iceberg, snow, etc., and substantially contains little impurities and hardly allows penetration and inhabitation of pathogens. The glacial snow water is characterized by a pH of 7.8 to 8.6, a density of 0.06 to 0.8 g/cm³, and an atomic size of about 20 Å (angstrom). A content of heavy water (D₂O) is about ¼ less than that of general water. Fresh water contains about 1/7000 of heavy water, while natural water is known to contain about 0.015% of heavy water. Heavy water (D₂O) has a molecular weight of 20.03, which is larger than general water, and also has about 10% higher density than general water. Heavy water (D₂O) is known to have strong inhibitory action on the metabolism of living organisms. Glacial snow has low deuterium content and a heavy water content of about 0.02% which is one quarter less than general water, whereby the glacial snow water is very suitable for activity of organisms.

### PRIOR ART DOCUMENT

### Patent Document

(Patent Document 1) Korean Patent Registration No. 10-0894834
(Patent Document 2) Korean Patent Laid-Open Publication No. 10-2005-0024872
(Patent Document 3) Korean Patent Laid-Open Publication No. 10-1997-0061105
(Patent Document 4) Korean Patent Publication 10-1284727
(Patent Document 5) Chinese Patent Laid-Open Publication No. CN104585573A
(Patent Document 6) Chinese Patent Laid-Open Publication No. CN105495615A

### DISCLOSURE

### Technical Problem

Pollen includes a lot of components such as essential nutrients and diverse active ingredients. However, the surface of the pollen is covered with a hard husk and the husk is not decomposed when eaten, and thus has very little utilization rate in the body (that is, “bioavailability”). In addition, pollen, flowers, honey glands of flowers, fruit and plant peels, etc., as well as many other natural substances commonly known to be useful for the human body involve substantially low bioavailability when applied to the human body. For this reason, only some of the active ingredients described above are used or exhibit substantially little efficacy. This is because an enzyme absorbing and degrading the active ingredients contained in natural substances does not exist or is not present in high enough concentration in the human body. In the case of fermentation, such natural substances are often degraded by microorganisms to thus increase bioavailability in the human body. In some cases, the natural substances may be converted into new effective ingredients during fermentation. The increase in bioavailability and/or the conversion into effective ingredients according to fermentation may significantly vary depending upon specific fermentation processes.

An object of the present invention is to provide a naturally fermented composition of pollen, which is rich in active ingredients and nutrients and has improved bioavailability, wherein the composition is obtained by natural fermentation of pollen-containing plant parts such as bee pollen, other pollen, edible flowers, etc. or the same admixed with miscellaneous pollen, fruits and fruit crops, vegetables, medicinal plants, fruit and plant peels, etc., using only 100% glacial snow water without addition of yeast or microorganisms, as well as a preparation method thereof. In addition, another object of the present invention is to provide antioxidant drink and food including the naturally fermented composition of pollen produced as described above.

### Technical Solution

As used herein, “glacial water” refers to water derived from “firn (or neve), glaciers, icebergs and snow, which is defined as water having pH 7.5 to 8.8 and a density of 0.05 to 0.85 g/cm³”.

As used herein, “pollen” is generally defined as including bee pollen except when it is specifically distinguished from bee pollen.

As used herein, “flower pollen” is pollen or a part of plants including pollen, and is defined as being selected from bee pollen, flower pollen, flowers, flower buds, petals, flower honey glands, etc.

In order to achieve the above purposes, the present invention may provide a naturally fermented composition of pollen, obtained by:
melting and filtering firn (or neve), glaciers, icebergs and/or snow to prepare glacial snow water with pH 7.5 to 8.8 and a density 0.05 to 0.85 g/cm³;
mixing the prepared water with at least one pollen selected from bee pollen, flower pollen, flowers, flower buds, petals, and flower honey glands, and
fermenting the mixture for 3 months or more without further inoculation of microorganisms.

When the pollen is mixed with the glacial snow water, one or more plants selected from pollen of mixed flowers, fruits and fruit products, vegetables, medicinal plants, fruit and plant peels, stevia dried leaves, and dried herbs, stems and roots may be further mixed.

Further, the present invention may provide a method for preparation of a naturally fermented composition of pollen using glacial snow water, which includes:
melting and filtering firn (or neve), glaciers, icebergs and/or snow to prepare glacial snow water with pH 7.5 to 8.8 and a density 0.05 to 0.85 g/cm³, and mixing the prepared water with at least one pollen selected from bee pollen, flower pollen, flowers, flower buds, petals, and flower honey glands, and
fermenting the mixture produced in the above step for 3 months or more without further inoculation of microorganisms.

In addition, the present invention may provide antioxidant drink and food including the naturally fermented composition of pollen using the glacial snow water obtained as described above.

### Advantageous Effects

As described above, the present invention provides a naturally fermented composition obtained by naturally fermenting pollen with only 100% glacial snow water without further addition of yeast or microorganisms, so that the composition is rich in active ingredients and nutrients such as essential amino acids and vitamins, wherein a variety of ingredients contained in the pollen have been suitably fermented for desired use in the human body. In particular, the outer skin of pollen may be dissolved by only natural fermentation, and about 200 species of nutrients and active ingredients contained in the pollen may be fermented to be easily usable in the human body and may be directly introduced to the composition. The naturally fermented composition of pollen according to the present invention may have increased effective ingredients and efficacies through fermentation as compared to the original material, and may greatly improve bioavailability in the human body. Furthermore, the composition of the present invention can be fermented and preserved without harmful components such as artificial additives or preservatives.

The naturally fermented composition of pollen obtained according to the present invention is substantially produced by a process for natural fermentation of plant components. Therefore, like cognac (brandy), whiskey, wine and the like, the composition of the present invention may ripen and be stored for decades.

The naturally fermented composition of pollen according to the present invention may include abundant nutrients and active ingredients and can exhibit diverse effects, in particular, antibacterial effect, anti-inflammatory effect, antioxidant effect, anti-aging effect, whitening effect, etc. As demonstrated in the examples of the present invention, excellent antioxidant effects are achieved.

### BEST MODE

The naturally fermented composition of pollen obtained using glacial snow water according to the present invention is substantially produced by mixing pollen with glacial snow water and then naturally fermenting the mixture.

The pollen is pollen, bee pollen or a portion of a plant including pollen. The bee pollen or pollen is not particularly limited and is preferably the bee pollen or the pollen derived from any one of edible flowers, mixed flowers, wildflowers, fruits and fruit products, vegetables and medicinal plants. The bee pollen or pollen used herein may be natural pollen with the husk taken from nature.

A portion of the plant including the pollen is preferably any one among flowers, buds, petals, and honey glands of flowers. More preferably, any one of edible flowers, buds, petals and honey glands of flowers is used.

The flower honey glands may include, but are not particularly limited to, every honey gland obtainable from flowers. Preferably, the flower honey gland is obtainable from any of edible plants including fruit trees, medicinal plants, mixed flowers, wildflowers, edible flowers and vegetables.

Preferably, the pollen is mixed at a ratio of 1 to 100 g based on dry weight with respect to 1 L of glacial snow water. More preferably, the pollen is mixed at a ratio of 2 to 70 g and, particularly preferably, at a ratio of 5 to 40 g based on dry weight with respect to 1 L of glacial snow water.

When mixing the glacial snow water and pollen, one or more plants selected from pollens of mixed flowers, fruits and fruit products, vegetables, medicinal plants, fruit and plant peels, stevia dried leaves, and dried herbs, stems and roots may be further mixed.

The pollen and the plants described above are preferably mixed in a weight ratio of 1:1 to 100, more preferably 1:2 to 50.

The plants are preferably dried and mixed with the glacial snow water.

When mixing the glacial snow water and pollen, natural plants may be optionally added to give taste, aroma, acidity, etc. if desired.

The glacial snow water is water obtained by melting and filtering firn (or neve), glacier snow, iceberg, snow, etc., which is weak-alkaline water having pH of 7.5 to 8.8, a density of 0.05 to 0.85 g/cm³ and an atomic size of about 20 Å (angstrom). More preferably, this is weak-alkaline water having pH of 7.8 to 8.6 and a density of 0.06 to 0.8 g/cm³.

Such glacial snow water has specific properties and efficacies different from those of general water. The glacial snow water such as firn (or neve) is characterized in that, when snow is strongly cooled and solidified by direct solidification and recrystallization, very little other gases are incorporated into an ice-crystal structure, whereby the structure is tight and dense, and has low surface tension and about ¼ less content of heavy water (D₂O) as compared to general water. Further, the glacial snow water includes a lot of enzymatic compounds, especially, colloidal inorganic particles, which in turn increases energy by interaction with structural pressure inside molecules. Further, the glacial snow water is similar to cell fluids of an organism (animals and plants) to thus be easily absorbed by living organisms, while including very little impurities. In addition, the glacial snow water is rich in active hydrogen to prevent and remove active oxygen, and has a strong ice-crystal structure through which pathogens or viruses cannot penetrate, thereby promoting the metabolism of cells to prevent ageing.

Useful microorganisms and enzymes such as yeast are present on pollen, flower buds, honey glands of flowers and the surface of fruit, and are rich in various minerals including high quality amino acids, nucleic acids, fatty acids, hormones, vitamins and zinc. In addition, unlike general water, the glacial snow water includes many enzymatic compounds, particles of colloidal inorganic compounds, and low water content and a low density of heavy water (D₂O)). For this reason, natural fermentation may be performed without addition of other microorganisms, yeasts, enzymes, etc. because of the action of useful microorganisms and enzymes present in the mixture when the glacial snow water and the plant components are mixed. In addition, the nutrients and minerals contained in the mixture may support the action of enzymes and microorganisms to proceed with optimized fermentation and enzyme chemical reactions. Further, the characteristics of the glacial snow water described above may function to promote the activity of an organism when the organism is introduced, so that the glacial snow water in contact with the yeast and/or enzyme present in pollen, flower buds, honey glands of the flower, the surface of fruit, etc. as well as interaction between both of the water and the yeast and/or enzymes described above may facilitate and catalyze the reaction. Further, in this process, activity of dehydrogenase in the glacial snow water is increased so that protons (H⁺) may quickly move and a structure of water molecule is dense due to a high ratio of hexagonal structures, thereby inducing high activity of microorganisms during fermentation.

Further, a molecular structure of the plants is clustered into a low-molecular structure, which in turn allows various nutrients contained in bee pollen and flower buds to be easily absorbed by the body. In addition, the glacial snow water is rich in active hydrogen to prevent and remove active oxygen, and has a strong ice-crystal structure through which pathogens or viruses cannot penetrate, whereby the naturally fermented composition of pollen according to the present invention obtained through fermentation does not decay or is not deteriorated even if it is stored for a prolonged period of time.

In particular, when the glacial snow water is mixed with pollen, flower buds or flower honey glands, the outer skin with a hard structure of the pollen is easily dissolved by enzymatic reaction, and various components, enzymes and/or microorganisms contained in the pollen, flower buds or flower honey glands are subjected to active enzymatic reaction and microbial metabolism and are changed into low molecular weight molecules, thereby being easily absorbed into the body. Further, allergens contained in the outer skin of the pollen may also be degraded and thus overcome allergic problems. In addition, there is no unpleasant taste or smell when drinking.

The fermentation may be performed at 1 to 40° ° C. for 3 months or more, preferably, at 10 to 38° C. for 4 to 12 months and, more preferably, at 20 to 30ºC for 4 to 7 months.

However, this is only a minimum period necessary for commercialization and, even thereafter, the naturally fermented composition of pollen according to the present invention may be preserved for several decades. The naturally fermented composition of pollen according to the present invention is obtained by natural fermentation of plant components using only the glacial snow water. Like cognac (brandy), whiskey, wine, etc., the above composition does not decay or is not deteriorated and, therefore, can be stored for decades. Even like such liquor as listed above, the composition of the present invention is further matured (or ripened) during preservation so as to increase beneficial ingredients while improving taste and flavor.

A method for manufacturing the naturally fermented composition of pollen according to the present invention will be described in detail below. The manufacturing method according to the present invention may include mixing the glacial snow water with pollen and then fermenting the mixture. Hereinafter, the manufacturing method will be described by stages.

Mixing Glacial Snow Water and Pollen

Pollen is mixed with the glacial snow water, or other plant portions besides the pollen are further mixed with the glacial snow water. Description of pollen, glacial snow water and other plant portions is substantially the same as that of the above composition.

Fermentation

The fermentation step in the present invention is executed without introduction of any artificial microorganism, yeast, enzyme from the outside. The fermentation is performed at 1 to 40° C. for 3 months or more. Preferably, the fermentation is performed at 10 to 38° C. for 4 to 12 months and, more preferably, at 20 to 30ºC for 4 to 7 months. Such fermentation may be aerobic fermentation.

The naturally fermented composition of pollen obtained according to the present invention contains abundant nutrients and active ingredients, and exhibits excellent antioxidant effects. The naturally fermented composition of pollen according to the present invention may be utilized in food and beverage, in particular, can be used as an antioxidant beverage.

Specifically, pollen has long been used as a health functional food, and the naturally fermented composition of the pollen according to the present invention may also be produced as various formulations such as liquid formulations, granules, pills, tablets, capsules, and the like, and may be used as health food. In addition, the composition of the present invention may be contained in general foods to impart functional properties such as anti-oxidation to foods and to enrich the nutrition of foods. For example, the composition may be contained in confection, bread, rice cake, etc. and may also be contained in soy sauce, gochujang (Korean red pepper paste), soybean paste and soy sauce in order to impart functionality and enhance nutrition of food. Further, the composition may also be incorporated into existing beverages and various kinds of alcoholic drinks.

As used herein, “health food” is typically defined as food taken by a consumer who expects specific functions to accommodate useful effects for health purposes such as nutrient control or physiological action with respect to the structure and functions of the human body. More particularly, the health food defined above may include all meanings that are commonly called “health functional food”, “health food”, “functional food”, etc. without limitation to legal regulations.

As used herein, “food” includes the above-mentioned health food as well as general food.

### Preferred Embodiment of Invention

### Example

Hereinafter, the present invention will be described in more detail with reference to examples and experimental Examples. However, the following examples and experimental examples illustrate the present invention only but the scope of the present invention is not limited thereto.

### Example 1

Preparation of Naturally Fermented Composition of Pollen 120 g of dry bee pollen was mixed in 20 L of glacial snow water (about pH 8.0, a density of about 0.8 g/cm³) and fermented at room temperature of about 20 to 30° ° C. for 20 months to obtain a naturally fermented composition of pollen.

### Example 2

Preparation of Naturally Fermented Composition of Pollen

Except for using 120 g of dried Japanese apricot blossom pollen 120 g instead of bee pollen, a naturally fermented composition of pollen was prepared in the same manner as in Example 1.

### Example 3

Preparation of Naturally Fermented Composition of Pollen

120 g of dried Japanese apricot blossom pollen and 600 g of dried apple peel were mixed in 20 L of glacial snow water (about pH 8.2, a density of about 0.7 g/cm³) and fermented at room temperature of about 20 to 30° C. for 5 months to obtain a naturally fermented composition of pollen.

### Example 4

Preparation of Naturally Fermented Composition of Pollen

Except for 240 g of honey glands of dried pear flowers instead of 600 g of dried apple peel, a naturally fermented composition of pollen was prepared in the same manner as in Example 3.

### Experimental Example 1

Fermentation Comparison of Fermentation by Types of Water 120 g of dried bee pollen was mixed in 20 L of water, followed by fermentation at 25 to 30° C., and the fermentation was observed. Each of waters listed in Table 1 was used for experiment, and the glacial snow water having about pH 8.0 and a density of about 0.8 g/cm³ as in Example 1 was used. Experimental results are shown in Table 1 below.

As can be seen in Table 1, the mixture was not fermented but decayed in the general water such as groundwater, mineral water and bottled water, whereas fermentation proceeded well in glacial snow water.

TABLE 1

| | | | | | Enzyme | |
|---|---|---|---|---|---|---|
| | Day 15 | Day 25 | Day 35 | Day 45 | activity | Remark |
| Underground | None | None | None | Oxidative | ↓ | No |
| water | | | Odor | decay | | enzymatic |
| | | | occurrence | | | reaction |
| Mineral | None | None | ″ | Oxidative | ↓ | No |
| water | | | | decay | | enzymatic |
| | | | | | | reaction |
| Deep sea | A | Enzymatic | Low | No | → | A little |
| water | little | reaction | enzyme | enzyme | | enzymatic |
| | enzymatic | | activity | activity | | reaction |
| | reaction | | | | | but |
| | | | | | | stopped |
| | | | | | | shortly |
| Bottled | None | None | None | Oxidative | ↓ | No |
| water by | | | Odor | decay | | enzymatic |
| Company A | | | occurrence | | | reaction |
| Bottled | None | None | ″ | ″ | ↓ | No |
| water by | | | | | | enzymatic |
| Company B | | | | | | reaction |
| Bottled | None | None | ″ | ″ | ↓ | No |
| water by | | | | | | enzymatic |
| Company C | | | | | | reaction |
| Bottled | None | None | ″ | ″ | ↓ | No |
| water by | | | | | | enzymatic |
| Company D | | | | | | reaction |
| Bottled | None | None | ″ | ″ | ↓ | No |
| water by | | | | | | enzymatic |
| Company E | | | | | | reaction |
| Glacial | Enzymatic | Active | Continued | High | ↑ | Enzymatic |
| snow | reaction | | fermentation | enzyme | | reaction |
| water | | | | actiyity | | maintaining |
| | | | | | | optimized |
| | | | | | | status |

### Experimental Example 2

Ingredient Test

Ingredient test of the naturally fermented composition of pollen prepared in Example 1 was executed by the Jeollabuk-do Institute of Health and Environment on request. The test results are shown in Table 2 below.

TABLE 2

| | Ash | Crude | Crude | Sugar | Vitamin C |
|---|---|---|---|---|---|
| Sample | (g/100 g) | fat (%) | protein (%) | (g) | (mg/100 g) |
| Example | 0.0 | 0.0 | 0.03 | 0.0 | 0.62 |
| 1 | | | | | |

### Experimental Example 3

Quality Inspection

The naturally fermented composition of pollen prepared in Example 1 was subjected to quality inspection with test items listed in Table 3 below. Quality inspection was executed by Sunchang-gun Soy Sauce Office and the test results are shown in Table 3.

TABLE 3

| Test items | Result |
|---|---|
| Crude protein | 0.004% |
| pH | 7.92 |
| General bacterial count | Not detected |
| Amino acid nitrogen | Not detected |

### Experimental Example 4

Antioxidant Activity Assay

The naturally fermented composition of pollen prepared in Example 1 as a sample was analyzed for antioxidant activity by the electron donating ability analysis method (DPPH).

The test was executed by the Korea Medical Science Institute on request, and the test methods and results are as follows.

1. Test Method

1) 2 ml of sample was mixed with 2 ml of 0.4 mM DPPH (1,1-diphenyl-2-picryl hydrazyl) methanol solution,
followed by reaction at room temperature for 30 minutes.

2) After 30 minutes, absorbance at 517 nm was measured and the measured result was applied to the following equation to evaluate antioxidant activity.

$$DDPH Scavenging ( \% ) = \frac{C - ( {S}_{1} - {S}_{2} )}{C} \times 100$$

C: methanol+DPPH, S1: sample+DPPH, S2: sample+methanol

3) Methanol was used as a control.

2. Results

The results are shown in Table 4 below. The antioxidant activity of the naturally fermented composition of pollen according to the present invention was 37.9% which was demonstrated to be significantly high.

TABLE 4

| Sample | DPPH assay (%) |
|---|---|
| Naturally fermented | 37.9 |
| composition of pollen in | |
| Example 1 | |

### INDUSTRIAL APPLICABILITY

The naturally fermented composition of pollen according to the present invention contains abundant nutritional ingredients and active ingredients based on plants used for the same and may exhibit different effects, and therefore, may be used in a variety of applications, for example, medicines, cosmetics, foods, animal feeds, etc. In particular, the inventive composition may be used as foods such as health foods and beverages, antioxidant drinks, etc. In addition, the naturally fermented composition of pollen and the manufacturing method of the present invention are substantially new fermentation technologies which can be utilized in various bio-industries and food industries.

1. A method for preparation of a naturally fermented composition of pollen, comprising:
melting and filtering at least one selected from firn (or neve), glaciers, icebergs and snow to prepare glacial snow water with pH 7.5 to 8.8 and a density 0.05 to 0.85 g/cm3, and mixing the prepared glacial snow water with at least one pollen selected from bee pollen, flower pollen, flowers, flower buds, petals and flower honey glands; and
fermenting the mixture for 3 months or more without further inoculation of microorganisms.

2. The method according to claim 1, wherein the glacial snow water has pH 7.8 to 8.6 and a density of 0.06 to 0.8 g/cm3.

3. The method according to claim 1, wherein the pollen is mixed at a ratio of 1 to 100 g based on dry weight to 1 L of glacial snow water.

4. The method according to claim 3, wherein, when the pollen is mixed with the glacial snow water, one or more plants selected from pollen of mixed flowers, fruits and fruit products, vegetables, medicinal plants, fruit and plant peels, stevia dried leaves, and dried herbs, stems and roots is further mixed.

5. The method according to claim 3, wherein the fermentation is performed at 10 to 38° C. for 4 to 12 months.

6. The method according to claim 1, wherein, when the pollen is mixed with the glacial snow water, one or more plants selected from pollen of mixed flowers, fruits and fruit products, vegetables, medicinal plants, fruit and plant peels, stevia dried leaves, and dried herbs, stems and roots is further mixed.

7. The method according to claim 1, wherein the fermentation is performed at 10 to 38° C. for 4 to 12 months.

8. The method according to claim 2, wherein, when the pollen is mixed with the glacial snow water, one or more plants selected from pollen of mixed flowers, fruits and fruit products, vegetables, medicinal plants, fruit and plant peels, stevia dried leaves, and dried herbs, stems and roots is further mixed.

9. The method according to claim 2, wherein the fermentation is performed at 10 to 38° C. for 4 to 12 months.
